(19) United States
(12) Patent Application Publication
(10) Pub. No.: US 2013/0046219 A1
(43) Pub. Date: Feb. 21, 2013

(54) NEONATAL CRANIAL SUPPORT BONNET

(71) Applicants: Daniel Mendez, The Woodlands, TX (US); Israel Gonzalez Cruz, Cibolo, TX (US); Nicholas Flores, San Antonio, TX (US)

(72) Inventors: Daniel Mendez, The Woodlands, TX (US); Israel Gonzalez Cruz, Cibolo, TX (US); Nicholas Flores, San Antonio, TX (US)

(73) Assignee: Board of Regents of the University of Texas System, Austin, TX (US)

(21) Appl. No.: 13/642,034

(22) PCT Filed: Apr. 21, 2011
(86) PCT No.: PCT/US11/33458

§ 371 Date: Oct. 18, 2012

Related U.S. Application Data
(60) Provisional application No. 61/327,647, filed on Apr. 23, 2010.

(51) Int. Cl. A61F 5/00

(52) U.S. Cl. 602/17

(57) ABSTRACT

A neonatal cranial support bonnet is configured to prevent a premature child's head from deforming under the force of its own weight, because of underdeveloped cranial plates. The bonnet includes a thin cotton shell with contoured gel packs inside. The gel packs are configured to distribute weight around the skull and eliminate or reduce high-pressure points so as to maintain proper shape of the cranium. The bonnet is also configured to not interfere with development, and to not obstruct airways for breathing, so as to meet current Sudden Infant Death Syndrome prevention guidelines.

This patent application claims priority to and the benefit of and incorporates herein by reference U.S. Provisional Patent Application Ser. No. 61/327,647, filed Apr. 23, 2010.

### BACKGROUND

This specification relates to neonatal care and more particularly to a neonatal cranial support bonnet for use with preterm infants.

In the United States, approximately 12.5% of all children are born premature. The total percentage of children born premature or preterm also appears to be increasing along with the total number of overall births. Because of this, hospitals, and in particular neonatal intensive care units (NICUs) must handle a large and ever-increasing number of fragile preterm infants.

There exist in the prior art head protectors designed for use with preterm infants in incubators. But when preterm infants are moved from incubators to open bassinets, SIDS guidelines require the removal of such head protectors. A preterm infant in an open bassinet cannot have so much as a pillow or a soft mattress to cushion its head.

Common complications for preterm infants include cranial deformations, which occur in approximately 20% of all preterm infants. Cranial deformations include plagiocephaly, brachycephaly, and scaphocephaly. Plagiocephaly and other deformations are characterized by the molding of the head into an unusual shape, such as a parallelogram, and a persistent flattened spot on the back or one side of the head. Positional and deformational plagiocephaly is most commonly caused by the position in which the baby spends most of his or her time. In a recent study, researchers found that the infants who exhibited positional plagiocephaly had lower scores than typical infants in observational tests used to evaluate cognitive and motor development. The majority of plagiocephaly cases require corrective measures once the child becomes full-term. A known method of preventing plagiocephaly while the child is in the open bassinets includes manually rotating the infant from side to side. Helmets may be custom made by a neonatologist, and are very expensive.

### BRIEF DESCRIPTION OF THE DRAWINGS

FIG. 1 is a side, perspective, upper view of an embodiment of Applicants' orthosis.

FIG. 2a is a front, top, perspective view of the orthosis, both FIGS. 1 and 2 showing the orthosis on an infant's head.

FIG. 2b is a rear elevational view of the infant's head nested in the bonnet.

FIG. 3 is an exploded side upper perspective view of Applicants' orthosis showing the manner in which a pocket in the flexible shell receives a gel pack.

FIG. 4 is the view of FIG. 3 showing a different embodiment of Applicants' device, an embodiment having different ear pads than the embodiment illustrated in FIG. 3.

FIGS. 5 and 6 are front perspective upper and front perspective side upper views of the embodiment of gel pack of Applicants' device as it would rest snug against an infant's head.

FIG. 7 is an illustration of the flexible shell of an embodiment of Applicants' device in top plane view, through the cross-section shown in FIG. 2 as it would fit to an infant's head.

FIG. 8 illustrates a gel pack for an embodiment of Applicants' device, laid flat.

FIG. 9 is the view of FIG. 8 showing dimensions and general regions of the gel pack portion of an embodiment of Applicants' device.

FIGS. 10a and 10b is a rear, top cross-section view through a vertical seam of an embodiment of Applicants' gel pack as well as a view showing the seam when the gel pack is contoured to the head.

FIG. 11 is a rear view of the gel pack apart from the shell as it would be positioned on an infant's head.

FIG. 12 is a cross-sectional view of the gel pack, infant's head, and the support surface showing the manner in which the gel pack distributes the pressure.

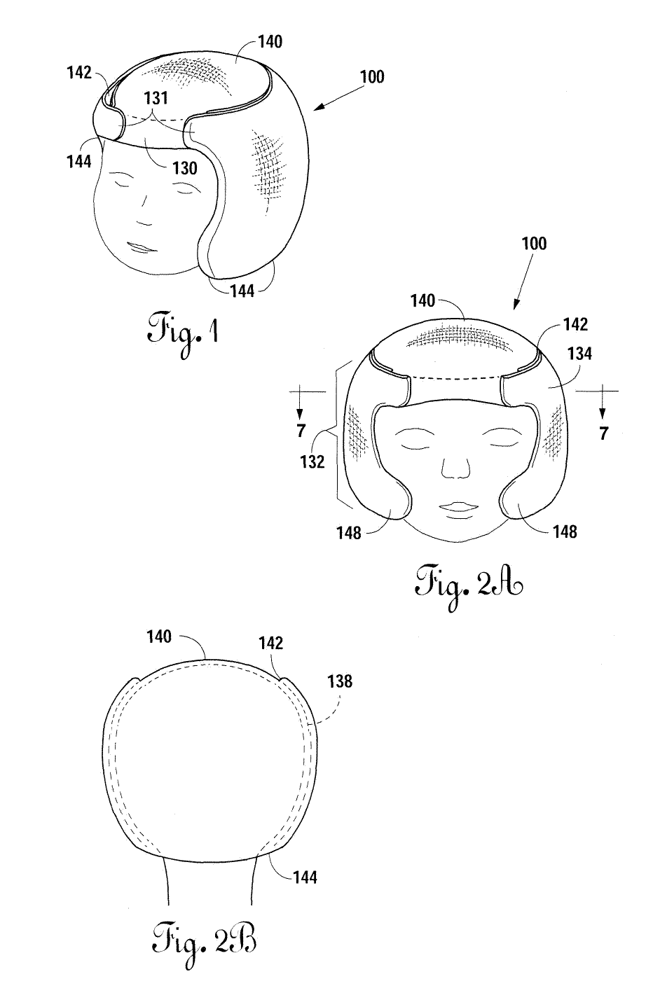

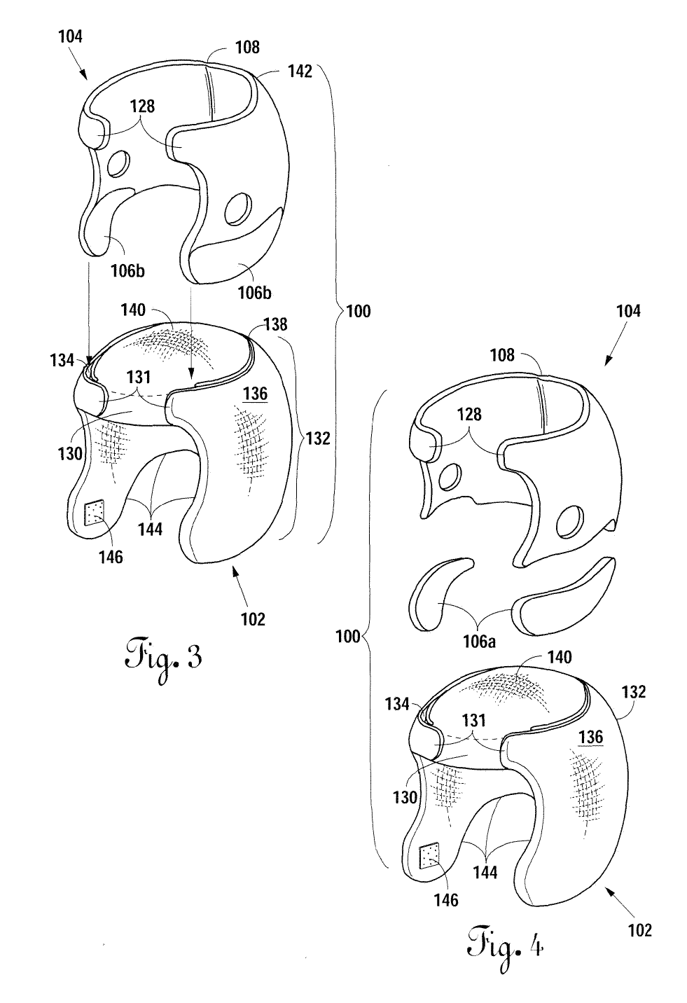

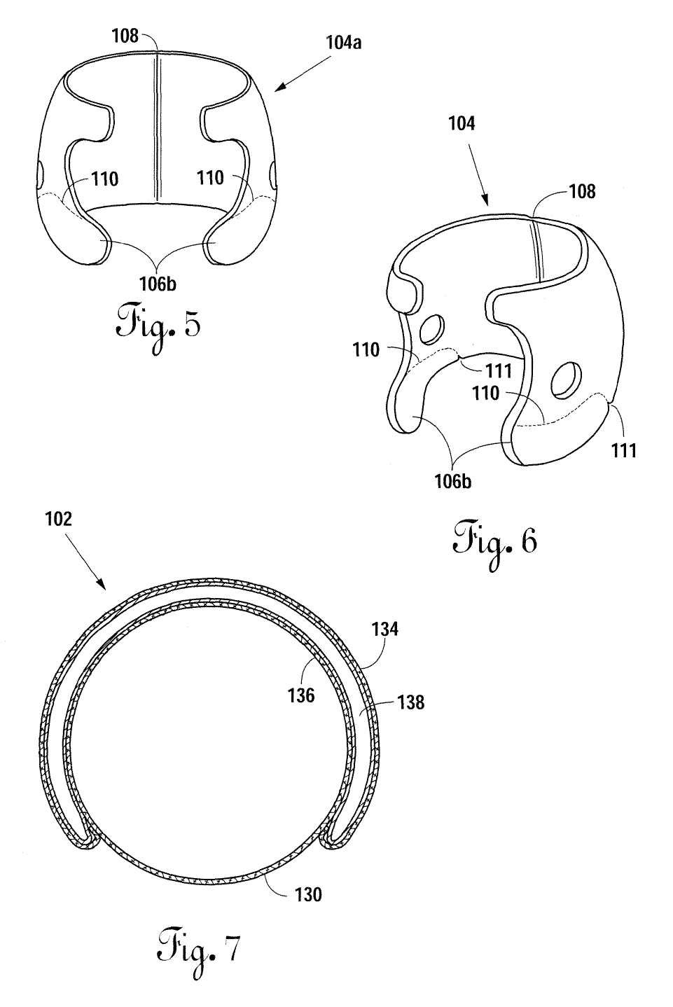

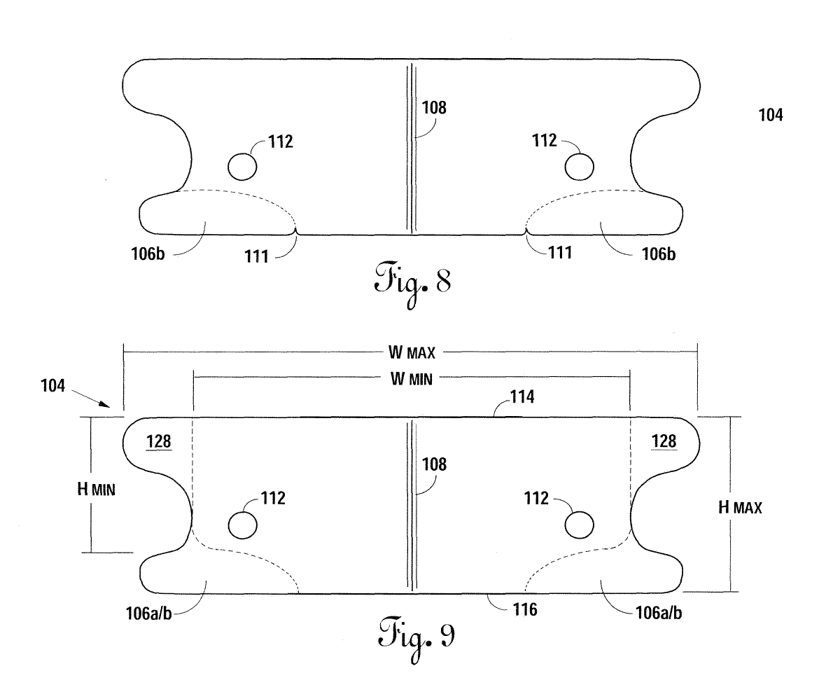

### SUMMARY OF THE INVENTION

In one aspect, a neonatal cranial support bonnet is configured to prevent a premature child's head from deforming under the force of its own weight, because of underdeveloped cranial plates. The bonnet includes a thin flexible fabric (for example, cotton) shell with contoured gel packs inside. The gel packs are configured to lay snuggly adjacent the infant's head to distribute weight around the skull and eliminate high-pressure points so as to help maintain proper shape of the cranium. Properly supporting the cranium can help to prevent so-called “flat head syndrome” and other deformation. The bonnet is also configured to be open-faced and to not interfere with development, and to not obstruct airways for breathing, so as to meet current SIDS prevention guidelines.

### DETAILED DESCRIPTION OF THE EMBODIMENTS

A neonatal cranial support bonnet can significantly reduce the problem of a premature or preterm infant's head being deformed under its own weight because of underdeveloped cranial plates. In particular, a neonatal cranial support bonnet may reduce point loads applied by the infant's head support surface, distribute pressure, and cradle the preterm infant's head to allow the cranial plates to develop properly and uniformly.

The neonatal cranial support bonnet of the present disclosure is configured to meet current guidelines for sudden infant death syndrome (SIDS) prevention. The bonnet is configured to distribute the pressure around the head of the preterm infant and eliminate or decrease points of high-pressure, thus reducing the tendency for the infants head to deform under its own weight. This may help to avoid the necessity of expensive and dramatic corrective measures that may be required to force a child's head back to normal shape once the preterm infant reaches full term.

A neonatal cranial support bonnet will now be described with more particular reference to the attached drawings.

The Figures illustrate the Applicants' orthosis 100 adapted to be received snugly against the head of an infant and to distribute pressure from the rear or either side of the baby's head depending on whether it is laying face up or with the head to one side or the other against a mattress or other support surface.

Applicants' orthosis 100 is seen to comprise a flexible shell 102 and a gel pack 104, the gel pack for being received within a pocket 138 of the flexible shell. Indeed, the gel pack itself is flexible and typically comprises a gel, such as polyethylene glycol gel. The gel may be water-based and include a suitable antiseptic or antibacterial substance.

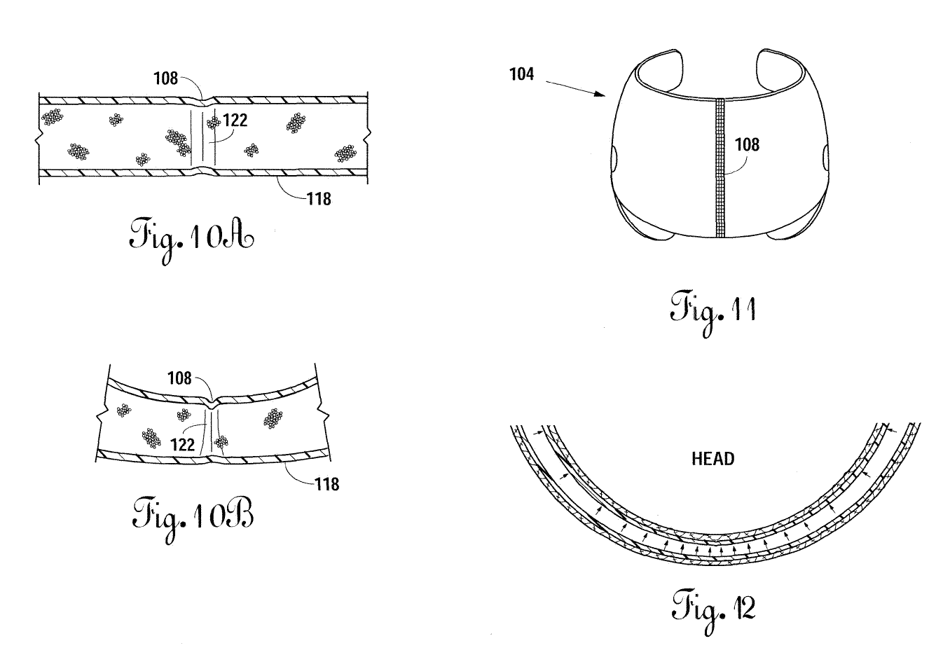

Turning now to the structure, construction and function of Applicants' gel pack 104, it may be seen to include optionally either non-gel (such as soft foam) ear pads 106a or gel ear pads 106b. Optimally, a vertical seam 108 is seen to be provided, which vertical seam allows the gel fluid to pass therethrough (see FIGS. 10a and 10b). That is to say, the vertical seam 108, as seen in FIG. 10a, may be comprised of a vertical members 122 engaging the top and bottom walls, located on the gel pack's interior allowing gel to flow therethrough as when an infant's head rests against a support surface. For gel jaw pads 106b, jaw pads seams 110 as well as a crease 111 may be provided, typically constructed also of mesh 122 or with non-permeable barrier. Seams 108/110 help provide shaping of gel pack 104 to infant's head, and help minimize buckling or folds on inner wall 120 as a tabular (rather than contoured) embodiment of gel pack, for example, as seen in FIG. 9, is shaped and placed into flexible shell 102, which is then slipped onto the infant's head and worn in the position as illustrated in the Figures. Creases 111 may be placed as indicated or other suitable places for ease of contouring the gel pack and for moving jaw pads to feed infant.

Ear openings or holes 112 are typically provided in gel pack 104 for avoiding constriction and pressure around the infant's ears, and for proper ventilation and proper auditory reception by the infant.

Gel pack 104, as set forth above, is flexible and has a skin, the skin typically formed into a top wall 114, bottom wall 116, wall 118, and inner wall 120 (see FIGS. 9 and 10a). The skin is typically flexible and non-porous to the gel, and may comprise urethane film or other suitable materials. One acceptable skin is RTV silicon about 0.15 mm in thickness. One gel is polyvinyl pyrrolidone cross-linked with carboxy vinyl. This is a water-based gel. A preferred viscosity range for pressure distribution is 30,000-200,000 centipose. A preferred viscosity is about 100,000 centipose.

As seen in FIG. 9, a view of the gel pack 104 laid flat, a top surface 118 is seen. Gel pack 104 may also include generally parallel top wall 114 and bottom wall 116. Inner wall 120 is spaced apart from wall 112, so the general thickness of the gel pack is uniform, typically about ¼″ to 1″, preferably about ½″.

Gel pack 104 is configured to, when received within flexible shell 102, which itself is dimensioned for snug receipt to an infant's head, cover substantially the following bones: occipital, at least the lower portions of the parietal bones, temporal bones, and any other cranial bones typically adjacent an infant's head when the infant is laying down, for example, on its side or back. Such coverage may be provided with a generally tabular flat laying or a contoured gel pack 104. For example, a flat laying (before insertion into the pocket) gel pack illustrated, in FIG. 9, is seen to have at least a width minimum and a width maximum in the general range of about 20 cm-about 50 cm, and a height minimum to a height maximum in the general range of about 5 cm-about 15 cm. These ranges would typically provide for small, medium, and large gel packs to fit in small, medium or large flexible shells 102, which flexible shells are configured to be snugly received against the head of an infant.

Turning to the flexible shell 102, it is seen to have an elastic band 130 that may be provided across forehead extensions 131. Elastic band 130 may undergo a color change as it stretches (baby's head growing), which color change may be indicative of a need to go to a larger bonnet. Optional forehead extensions 131 of shell 102 receive forehead extensions 128 of gel pack 104 for snug receipt into the pocket 138 of flexible shell 102. Flexible shell 102 is comprised of pocket portion 132 and a crown portion 140. Crown portion 140 may be a single fabric sheet or single member, and pocket portion 132 typically has an outer member 134 and an inner member 136, the outer and inner members defining the pocket 138. Pocket 138 is designed to snugly receive gel pack 104 thereinto and has an upper pocket opening 142 and a closed perimeter seam 144. Upper pocket opening may be configured, by a top edge of outer member standing above the gel pack 104, when inserted and seated in pocket 138, with Velcro tape or other removable fastener to seal and locate the gel pack within the pocket. Double sided tape 146 may be provided on jaw pad portions 148 of the shell to seal it to the infant's jaw.

The interior volume defined by the skin of the gel pack in the preferred embodiment presents a single cell. That is to say, in a preferred embodiment, the gel may flow unrestricted within the walls (skin) of the gel pack 104. There are no non-porous internal barriers to restrict the flow of the gel. Indeed, if gel jaw pads are used, then typically the unrestricted flow may even flow to the jaw pads. In an alternate preferred embodiment, with soft, flexible foam (non-gel) jaw pads, the jaw pads are not part of the gel pack, but are simply inserted into the pocket prior to inserting the gel pack and lay adjacent the gel pack snuggly against the infant's jaw.

The jaw pads will typically be connected by a seam or be unconnected to the gel pack (but in the pocket) and will allow the nurse to hold the infant and, with the bonnet 100 snuggly and properly positioned on the infant's head, to move the jaw pads out of the way for holding the infant's jaw while feeding the infant. Typically, dual-sided tape may be applied in the positions illustrated to help maintain the position of the bonnet and jaw pads on the infant's head during use. The double-sided tape will allow gentle removal of the jaw pads for infant feeding.

A single flexible gel pack may be used, in an alternate embodiment, without the flexible shell, and the means to attach the pack to the infant's head in the proper position may be several. There may be simply an elastic band or elastic sleeve (similar to a headband used by skiers) to slip over the shell to hold it against the infant's head, which elastic sleeve will have a portion coming across the forehead and may be contoured to the cranium and may cover the infant's ears and crown. In addition, the single gel pack may be taped, for example, with double-sided tape, to the infant's head, which tape may be removed and repositioned as the infant grows and as the head gets larger. Indeed, in place of the elastic band, which is illustrated in use with the flexible shell, there may be, instead, a two-piece Velcro band, each piece attached to the forehead extensions. By using a two-piece Velcro band, as the infant's head grows, the Velcro can be released and reattached so the two-piece band is slightly longer. Further, adhesive tape, including double-sided adhesive tape, may be used anywhere to attach either a single flexible gel pack directly to the infant's head or in conjunction with the shell, wherein the double-sided tape would lay between the fabric of the shell and the infant's skull anywhere the infant's skin contacts the shell and that is suitable to properly position the bonnet to the infant's head and to avoid slippage.

While a preferred embodiment includes a single gel pack, alternate embodiments may include multiple separate gel packs, including pockets, one or more configured to receive the multiple gel packs (either separately or as a group). These separate gel packs may be one gel pack for substantially covering the cranium at the rear of an infant's head where the rear of the head contacts the support surface when the infant is sleeping face up, and two separate gel packs, one for each side of the infant's head where the infant's head lays against the support surface when the infant is laying on one side or the other.

The shell may have the pocket on the outside or the inside. The gel pack or gel packs may come in multiple sizes, for example, small, medium, and large, to cover the infant's head, the various sizes of an infant's head as the infant grows. The shell comes in various sizes also.

To vary the amount of flexibility of the gel pack, the pressure of the gel therein may be increased. Typically, the gel is under sufficient pressure to give firmness, but flexibility to the gel pack, but not to prevent the gel pack from maintaining the contour of the infant's head, as set forth above. Viscosity of the gel substance within the gel pack may be varied, with greater viscosity providing for a smaller area of pressure distribution.

Turning to FIG. 12, it is seen that the gel pack is configured with gel pressure, gel viscosity, and pack thickness, or any other parameter or combination thereof such that the infant's head, wearing the orthosis and with the infant laying against the support surface, typically the outer and inner walls will not touch. Rather, pressure will be distributed, assisted by gel flow, away from the contact point against the support surface and therefore the pressure at the point of contact will be lessened as it is distributed as indicated by the arrows in FIG. 12.

While the subject of this specification has been described in connection with one or more exemplary embodiments, it is not intended to limit the claims to the particular forms set forth. On the contrary, the appended claims are intended to cover such alternatives, modifications and equivalents as may be included within their spirit and scope.

1. An orthosis for an infant's head, the orthosis comprising:
a flexible shell, the flexible shell for substantially covering the cranial bones of the infant's head while leaving the face substantially uncovered, the flexible shell having a pocket therein, the pocket configured to receive a gel pack, the pocket configured to lay adjacent at least some of the occipital bone of the infant's head, some of the temporal bones, some of the lower portion of the parietal bones, and part of the frontal bones; and
a gel pack having a resilient, flexible outer skin defining an interior portion, and having a gel substance having a viscosity, the gel substance filling the interior portion, the gel pack configured for snug receipt into the pocket of the flexible shell, the gel pack further configured to substantially cover at least the occipital bone of the infant's head, some of the temporal bones, some of the lower portion of the parietal bones and part of the frontal bones while leaving substantially uncovered the crown of the head, the ears and infant's face.

2. The orthosis of claim 1, wherein the gel substance has a viscosity of between 30,000 and 200,000 cp.

3. The orthosis of claim 2, wherein the gel substance has a viscosity of about 100,000 cp.

4. The orthosis of claim 1, wherein the gel substance is a water based gel.

5. The orthosis of claim 1, wherein the skin is a flexible silicon-based

6. The orthosis of claim 1, wherein the flexible shell is made of a soft, textile material.

7. The orthosis of claim 1, wherein the flexible shell includes an elastic band, Velcro or tape configured to cover part of the infant's forehead.

8. The orthosis of claim 7, wherein the elastic of the elastic band displays color variations as it is stretched.

9. The orthosis of claim 1, further including jaw pads.

10. The orthosis of claim 1, wherein the interior volume of the gel pack is comprised of a single cell.

11. The orthosis of claim 1, further including jaw pads wherein the jaw pads include adhesive to removably hold them against the infant's head.

12. The orthosis of claim 1, wherein the gel pack includes one or more seams, the seams configured to allow the gel to flow therethrough.

13. The orthosis of claim 1, comprising:
a flexible shell, the flexible shell for substantially covering the cranial bones of the infant's head while leaving the face substantially uncovered, the flexible shall having a pocket therein, the pocket configured to receive a gel pack, the pocket configured to lay adjacent at least some of the occipital bone of the infant's head, the temporal bones, the lower portion of the parietal bones, and part of the frontal bones; and
a gel pack having a resilient, flexible outer skin defining an interior portion, and a gel substance having a viscosity, the gel substance filling the interior portion, the gel pack configured for snug receipt into the pocket of the flexible shell, the gel pack further configured to substantially cover at least the occipital bone of the infant's head, the temporal bones, the lower portion of the parietal bones and part of the frontal bones while leaving substantially uncovered the crown of the head and part of the frontal bones, the ears and infant's face.
wherein the gel substance has a viscosity of between 30,000 and 200,000 cp;
wherein the gel substance is a water based gel;
wherein the skin is a flexible silicon-based material;
wherein the flexible shell includes a band configured to cover part of the infant's forehead;
further including jaw pads which include adhesive to removably hold them against the infant's head; and
wherein the interior volume comprises a single cell.

14. An orthosis for an infant's head comprising:
a single flexible gel pack configured to cover most of the cranial bones of an infant's head; and
means to removably secure the gel pack to the infant's head while leaving the face substantially uncovered.

15. The orthosis of claim 13, wherein means to removably secure the gel pack to the infant's head includes a flexible shell.

16. The orthosis of claim 14, wherein the flexible shell includes a pocket configured to receive the gel pack.

17. The orthosis of claim 15, further including jaw pads.

18. The orthosis of claim 14, wherein the flexible shell includes an adjustable band adapted to lay adjacent the infant's forehead.

19. The orthosis of claim 14, wherein the gel pack includes a gel having a viscosity of between 30,000 and 200,000 cps; and wherein the means to removably secure the gel pack to the infant's head is tape.
